(19) United States
(12) Patent Application Publication
(10) Pub. No.: US 2022/0296744 A1
(43) Pub. Date: Sep. 22, 2022

(54) Disinfectant Light Autonomous Robot Apparatus

(71) Applicant: Kenneth Thibodeaux, Napoleonville, LA (US)

(72) Inventor: Kenneth Thibodeaux, Napoleonville, LA (US)

(21) Appl. No.: 17/204,584

(22) Filed: Mar. 17, 2021

(51) Int. Cl. A61L 2/10; A61L 2/24; A61L 2/26

(52) U.S. Cl. CPC A61L 2/10; A61L 2/24; A61L 2/26; A61L 2202/11; A61L 2202/14; A61L 2202/16; A61L 2202/25

(57) ABSTRACT

A disinfectant light autonomous robot apparatus for sanitizing rooms with ultraviolet light includes a body having a body top side, a body bottom side, and a body perimeter. The body bottom side has a plurality of wheel cavities. A plurality of wheels is coupled within the plurality of wheel cavities. A drive motor is coupled within the body and is in operational communication with the plurality of wheels. A central processing unit is in operational communication with the drive motor. A plurality of sensors allows for automated maneuvering of the apparatus throughout a room. A battery is coupled within the body and is in operational communication with the central processing unit. An arm is pivotably coupled to the body top side and a plurality of ultraviolet lights is coupled to the arm. A touchscreen is coupled to the body to program the apparatus.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to disinfectant robot devices and more particularly pertains to a new disinfectant robot device for sanitizing rooms with ultraviolet (UV) light. The present device includes a foldable and extendable light arm with multiple 360° light heads. It also includes ball wheels and a circular robotic base for optimal maneuverability.

### (2) Description of Related Art Including Information Disclosed Under 37 CFR 1.97 and 1.98

The prior art relates to disinfectant robot devices. These devices often lack ultraviolet lights and particularly lack multiple 360° light sources at different heights. Known devices also lack round wheels to allow for a 0° turning radius.

### BRIEF SUMMARY OF THE INVENTION

An embodiment of the disclosure meets the needs presented above by generally comprising a body having a body top side, a body bottom side, and a body perimeter. The body bottom side has a plurality of wheel cavities. A plurality of wheels is coupled to the body. The plurality of wheels is coupled within the plurality of wheel cavities. A drive motor is coupled within the body and is in operational communication with the plurality of wheels. A central processing unit is coupled within the body. The central processing unit is in operational communication with the drive motor. A plurality of sensors is coupled to the body and is in operational communication with the central processing unit to allow for automated maneuvering of the apparatus throughout a room. A battery is coupled within the body and is in operational communication with the central processing unit. An arm is coupled to the body. The arm is pivotably coupled to the body top side and moves between a folded position parallel to the body top side and an extended position perpendicular to the body top side. A plurality of ultraviolet lights is coupled to the arm. Each of the ultraviolet lights is in operational communication with the central processing unit. A touchscreen is coupled to the body and is in operational communication with the central processing unit to program the apparatus.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a disinfectant light autonomous robot apparatus according to an embodiment of the disclosure.

FIG. 2 is an isometric view of an embodiment of the disclosure.

FIG. 3 is a side elevation view of an embodiment of the disclosure.

FIG. 4 is a rear elevation view of an embodiment of the disclosure.

FIG. 5 is a bottom plan view of an embodiment of the disclosure.

FIG. 6 is an in-use view of an embodiment of the disclosure.

FIG. 7 is an in-use view of an embodiment of the disclosure.

FIG. 8 is a block diagram view of an embodiment of the disclosure.

### DETAILED DESCRIPTION OF THE INVENTION

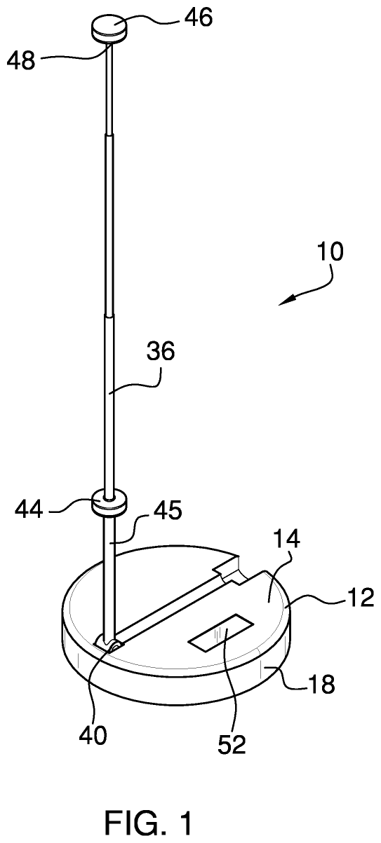

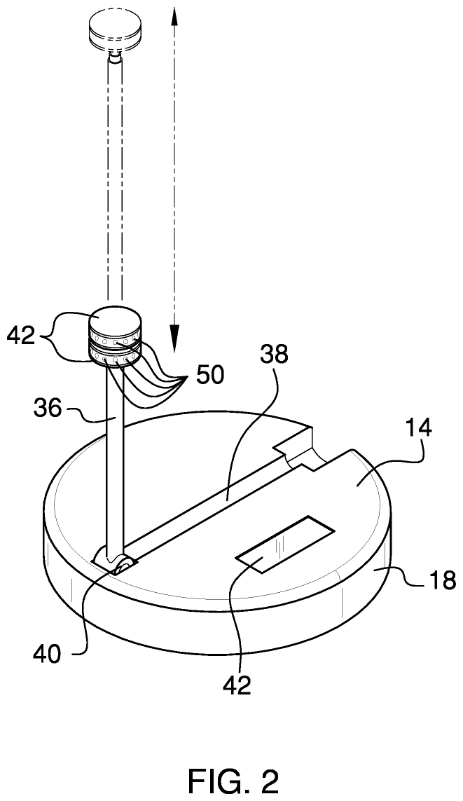

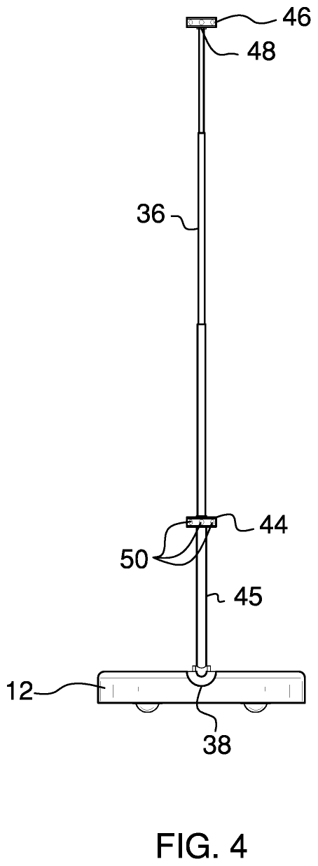

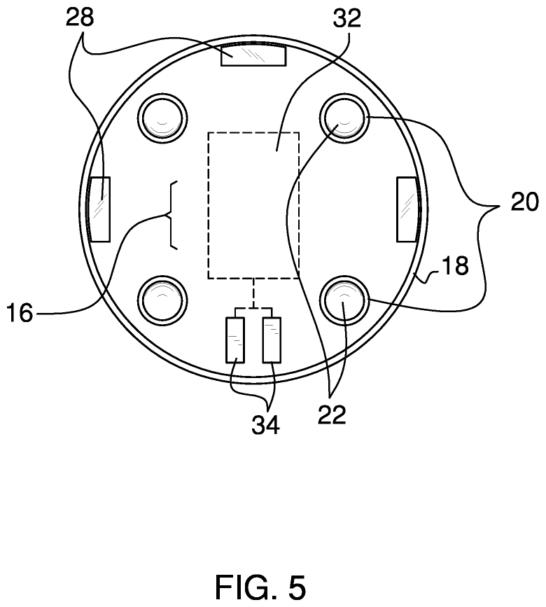

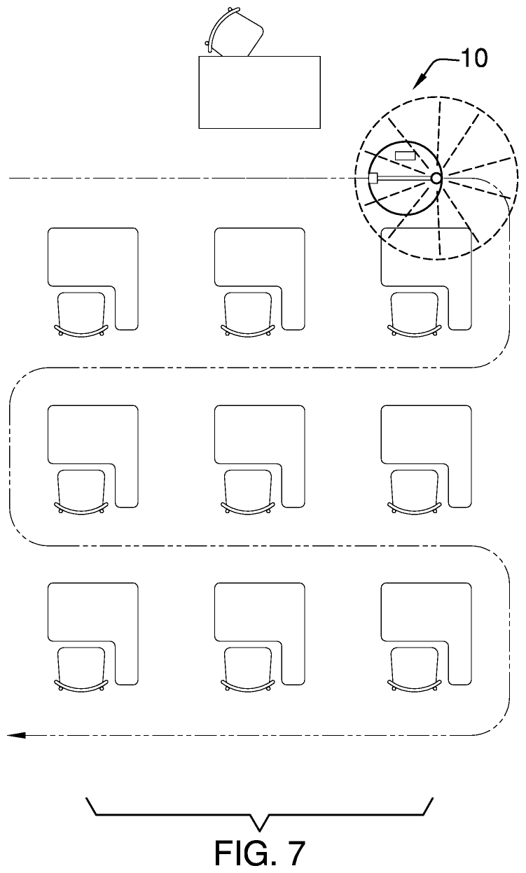

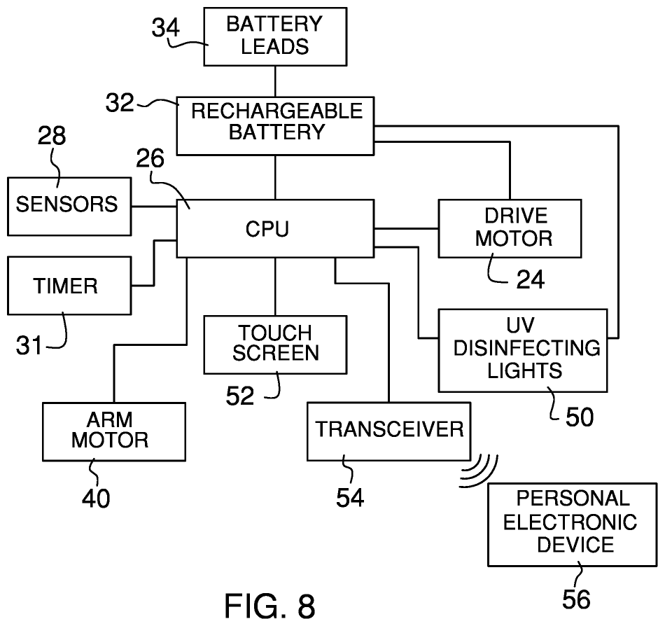

With reference now to the drawings, and in particular to FIGS. 1 through 8 thereof, a new disinfectant robot device embodying the principles and concepts of an embodiment of the disclosure and generally designated by the reference numeral 10 will be described.

As best illustrated in FIGS. 1 through 8, the disinfectant light autonomous robot apparatus 10 generally comprises a body 12 having a body top side 14, a body bottom side 16, and a body perimeter 18. The body bottom side 16 has a plurality of wheel cavities 20. The body 12 may be disc-shaped. A plurality of wheels 22 is coupled to the body 12. The plurality of wheels 22 is coupled within the plurality of wheel cavities 20. Each of the plurality of wheels 22 may be spherical to allow for a 0° turning radius and maximum maneuverability. A drive motor 24 is coupled within the body 12. The drive motor 24 is in operational communication with the plurality of wheels 22 to move the apparatus 10.

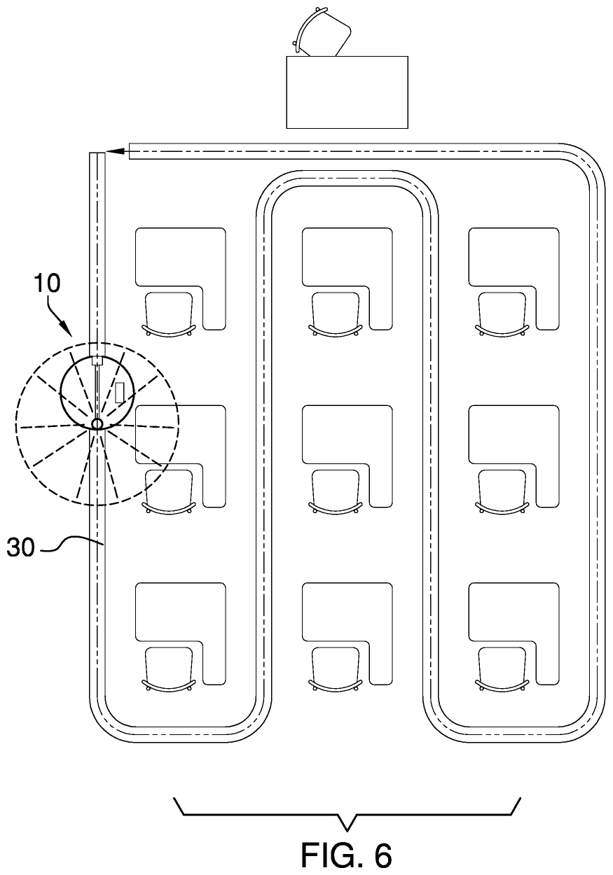

A central processing unit 26 is coupled within the body 12. The central processing unit 26 is in operational communication with the drive motor 24. A plurality of sensors 28 is coupled to the body 12. The plurality of sensors 28 is in operational communication with the central processing unit 26 to allow for automated maneuvering of the apparatus 10 throughout a room. The plurality of sensors 28 may be coupled to the body bottom side 16 adjacent the body perimeter 18. A tape track 30 may be placed on the floor to be detected by the plurality of sensors 28 for the apparatus 10 to follow a specific route as shown in FIG. 6. A timer 31 may be in operational communication with the central processing unit 26 for scheduled operation.

A battery 32 is coupled within the body 12. The battery 32 is in operational communication with the central processing unit 26. The battery 32 may have a pair of charging leads 34 coupled to the body bottom side 16.

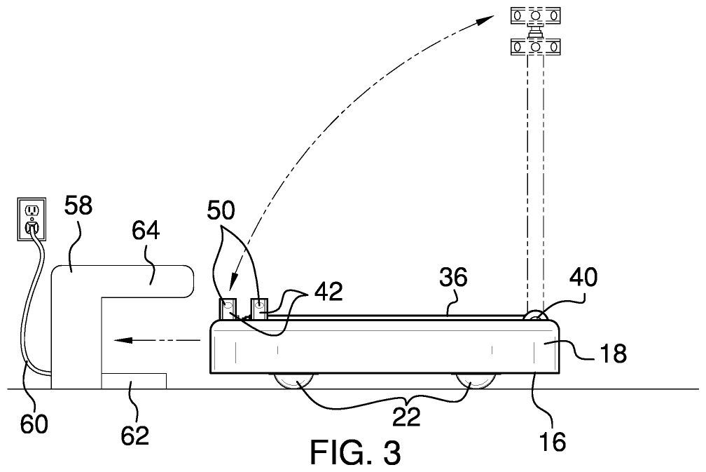

An arm 36 is coupled to the body 12. The arm 36 is pivotably coupled to the body top side 14 and moves between a folded position parallel to the body top side 14 and an extended position perpendicular to the body top side 14 as shown in FIG. 3. The arm 36 may be telescopable. The body top side 14 may have an arm channel 38 to selectively receive the arm 36 in the folded position. The arm channel 38 may receive half of the arm 36 in the folded position as seen in FIG. 3. An arm motor 40 may be coupled to the arm 36. The arm motor 40 is in operational communication with the central processing unit 26 and moves the arm 36 between the folded position and the extended position.

A pair of light housings 42 is coupled to the arm 36. Each of the light housings 42 may be cylindrical. The arm channel 38 may also accommodate half of each of the pair of light housings 42. A lower housing 44 of the pair of light housings is coupled to a lower portion 45 of the arm and an upper housing 46 of the pair of light housings is coupled to a distal end 48 of the arm. The distance between the lower housing 44 and the upper housing 46 thus changes as the arm 36 telescopes.

A plurality of ultraviolet lights 50 coupled to the pair of light housings 42. Each of the ultraviolet lights 50 is in operational communication with the central processing unit 26. The plurality of ultraviolet lights 50 is coupled within the pair of light housings 42 and positioned to shine 360° around each light housing 42.

A touchscreen 52 is coupled to the body 12. The touchscreen 52 may be coupled to the body top side 14. The touchscreen 52 is in operational communication with the central processing unit 26 to program the apparatus 10. A transceiver 54 may be coupled within the body 12 and in operational communication with the central processing unit 26. The transceiver 54 is configured to be in wireless communication with a personal electronic device 56 to allow for remote programming and operation. A docking station 58 has a power cord 60 and is in operational communication with the pair of charging leads 34 of the battery. The docking station 58 has a dock base 62 and a dock cover 64. The dock cover 64 extends over the plurality of ultraviolet lights 50 to provide protection.

In use, the apparatus 10 follows a preprogrammed path or autonomously navigates the room using the plurality of sensors 28. The plurality of ultraviolet lights 50 sanitize the room during this path. When complete or when the battery 32 needs to be charged, the apparatus 10 returns to the docking station 58 and the arm 36 collapses and folds to the folded position before entering to contact the pair of charging leads 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A disinfectant light autonomous robot apparatus comprising:
a body having a body top side, a body bottom side, and a body perimeter, the body bottom side having a plurality of wheel cavities;
a plurality of wheels coupled to the body, the plurality of wheels being coupled within the plurality of wheel cavities;
a drive motor coupled within the body, the drive motor being in operational communication with the plurality of wheels;
a central processing unit coupled within the body, the central processing unit being in operational communication with the drive motor;
a plurality of sensors coupled to the body, the plurality of sensors being in operational communication with the central processing unit to allow for automated maneuvering of the apparatus throughout a room;
a battery coupled within the body, the battery being in operational communication with the central processing unit;
an arm coupled to the body, the arm being pivotably coupled to the body top side and moving between a folded position parallel to the body top side and an extended position perpendicular to the body top side;
a plurality of ultraviolet lights coupled to the arm, each of the ultraviolet lights being in operational communication with the central processing unit; and
a touchscreen coupled to the body, the touchscreen being in operational communication with the central processing unit to program the apparatus.

2. The disinfectant light autonomous robot apparatus of claim 1 further comprising a pair of light housings coupled to the arm; the plurality of ultraviolet lights being coupled within the pair of light housings.

3. The disinfectant light autonomous robot apparatus of claim 2 further comprising each of the light housings being cylindrical; the plurality of ultraviolet lights being positioned to shine 360° around each light housing.

4. The disinfectant light autonomous robot apparatus of claim 2 further comprising the arm being telescopable; a lower housing of the pair of light housings being coupled to a lower portion of the arm and an upper housing of the pair of light housings being coupled to a distal end of the arm.

5. The disinfectant light autonomous robot apparatus of claim 1 further comprising the body top side having an arm channel to selectively receive the arm in the folded position.

6. The disinfectant light autonomous robot apparatus of claim 5 further comprising the arm channel receiving half of the arm in the folded position.

7. The disinfectant light autonomous robot apparatus of claim 1 further comprising an arm motor coupled to the arm, the arm motor being in operational communication with the central processing unit and moving the arm between the folded position and the extended position.

8. The disinfectant light autonomous robot apparatus of claim 1 further comprising each of the plurality of wheels being spherical.

9. The disinfectant light autonomous robot apparatus of claim 1 further comprising the battery having a pair of charging leads coupled to the body bottom side; a docking station having a power cord and being in operational communication with the pair of charging leads.

10. The disinfectant light autonomous robot apparatus of claim 9 further comprising the docking station having a dock base and a dock cover, the dock cover extending over the plurality of ultraviolet lights.

11. A disinfectant light autonomous robot apparatus comprising:
a body having a body top side, a body bottom side, and a body perimeter, the body bottom side having a plurality of wheel cavities;
a plurality of wheels coupled to the body, the plurality of wheels being coupled within the plurality of wheel cavities, each of the plurality of wheels being spherical;
a drive motor coupled within the body, the drive motor being in operational communication with the plurality of wheels;
a central processing unit coupled within the body, the central processing unit being in operational communication with the drive motor;
a plurality of sensors coupled to the body, the plurality of sensors being in operational communication with the central processing unit to allow for automated maneuvering of the apparatus throughout a room;
a battery coupled within the body, the battery being in operational communication with the central processing unit, the battery having a pair of charging leads coupled to the body bottom side;
an arm coupled to the body, the arm being pivotably coupled to the body top side and moving between a folded position parallel to the body top side and an extended position perpendicular to the body top side, the arm being telescopable, the body top side having an arm channel to selectively receive the arm in the folded position, the arm channel receiving half of the arm in the folded position;
an arm motor coupled to the arm, the arm motor being in operational communication with the central processing unit and moving the arm between the folded position and the extended position;
a pair of light housings coupled to the arm, each of the light housings being cylindrical, a lower housing of the pair of light housings being coupled to a lower portion of the arm and an upper housing of the pair of light housings being coupled to a distal end of the arm;
a plurality of ultraviolet lights coupled to the pair of light housings, each of the ultraviolet lights being in operational communication with the central processing unit, the plurality of ultraviolet lights being coupled within the pair of light housings positioned to shine 360° around each light housing;
a touchscreen coupled to the body, the touchscreen being in operational communication with the central processing unit to program the apparatus; and
a docking station having a power cord and being in operational communication with the pair of charging leads, the docking station having a dock base and a dock cover, the dock cover extending over the plurality of ultraviolet lights.
